(19) United States
(12) Patent Application Publication
(10) Pub. No.: US 2009/0297576 A1
(43) Pub. Date: Dec. 3, 2009

(54) Local Delivery of PAR-1 Antagonists to Treat Vascular Complications

(75) Inventors: Ayala Hezi-Yamit, Santa Rosa, CA (US); Jennifer Wong, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 12/131,606

(22) Filed: Jun. 2, 2008

(51) Int. Cl. A61K 9/00; A61K 31/443; A61K 31/56; A61K 33/00; A61K 31/662; A61K 39/395; A61K 31/7052; A61K 31/4353; A61P 9/00

(52) U.S. Cl. 424/423; 514/337; 514/182; 424/718; 514/108; 424/130.1; 514/291; 514/44 R; 514/44 A

(57) ABSTRACT

Described herein are methods and medical devices used to deliver bioactive agents locally to patients in need of treatment and/or prevention of cardiovascular conditions Local delivery of protease-activated receptor 1 (PAR-1) antagonists are described herein from implantable medical devices including, but not limited to, stents.

### FIELD OF THE INVENTION

The present invention relates to the local delivery of PAR-1 antagonists to treat vascular conditions.

### BACKGROUND OF THE INVENTION

Cardiovascular disease is a leading cause of morbidity and mortality. Major cardiovascular complications include aneurysm and stenosis. Both conditions can be treated using the methods of angioplasty and/or stenting. Both procedures commonly involve the deployment of a stent using a catheter into the effected region of vasculature, thereby re-structuring or re-enforcing the existing vasculature.

On occasion, following either of the above procedures, complications may arise. Two common side-effects of the stenting procedure are restenosis and in-stent thrombosis. Restenosis involves the re-occlusion of the vessel which was treated following a stenting procedure. Thrombosis occurs when a clot forms as a result of the stenting. Both conditions result in reduced blood flow through the effected region.

Typically, a common method of treatment of either of these side-effects involves the systemic administration of bioactive agents to reduce processes such as, but not limited to, smooth muscle cell proliferation, formation of extra cellular matrix, and inflammation. One problem with systemic administration of drugs such as anti-inflammatories, matrix metalloproteinase inhibitors, and anti-proliferatives are their side-effects and toxicity. Methods need to be developed enabling site specific delivery of bioactive agents capable of treating restenosis and in-stent thrombosis to the effected vasculature.

### SUMMARY OF THE INVENTION

Described herein are methods and medical devices used to deliver bioactive agents locally to vasculature in need thereof. Devices and methods described herein can be useful in treating and/or preventing cardiovascular conditions including, but not limited to, restenosis, in-stent restenosis, thrombosis and in-stent thrombosis. Protease-activated receptor 1 (PAR-1) antagonists can be useful in preventing and treating thrombosis and/or restenosis without the side effects of systemic delivery by local delivery via an implantable medical device. In one embodiment, the medical device is a stent and the PAR-1 antagonist is SCH-530348.

### DEFINITION OF TERMS

Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578), as well as other rapamycin hydroxyesters.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Nonbiodegradable: As used herein “nonbiodegradable” refers to a polymeric composition that is biocompatible and not subject to being broken down in vivo through the action of normal biochemical pathways.

Not Substantially Toxic: As used herein “not substantially toxic” shall mean systemic or localized toxicity wherein the benefit to the recipient is out-weighted by the physiologically harmful effects of the treatment as determined by physicians and pharmacologists having ordinary skill in the art of toxicity.

Pharmaceutically Acceptable: As used herein “pharmaceutically acceptable” refers to all derivatives and salts that are not substantially toxic at effective levels in vivo.

### DETAILED DESCRIPTION OF THE INVENTION

Methods and devices are described herein for the local delivery of bioactive agents useful in the treatment and/or prevention of restenosis and/or in-stent thrombosis. Thrombin plays a significant role in both restenosis and in-stent thrombosis. Thrombin plays a key role in the initiation of in-stent thrombosis by triggering platelet activation. In addition, thrombin prompts restenosis via a direct effect in enhancing proliferation and migration of smooth muscle cells.

One method of reducing restenosis and in-stent thrombosis is to reduce the effects of thrombin itself. Protease-activated receptor 1 (PAR-1) is the cellular thrombin receptor which mediates the effects of thrombin on platelets and smooth muscle cells. Thus, inhibition of PAR-1, by a PAR-1 antagonist can be beneficial for reduction of restenosis and/or in-stent thrombosis. In such a case, the inventors have proposed the local delivery of a PAR-1 antagonist from an implantable medical device.

PAR-1 receptor antagonists function to inhibit the activity associated with the activation of the PAR-1 receptor. Thrombin binds to PAR-1 receptors where it functions to initiate the proliferation and/or migration of smooth muscle cells. The problem with using systemic PAR-1 antagonists to treat vascular complications is that the amount of bioactive agent necessary for treatment would vastly increase the risk of undesired systemic side effects, to the point of being toxic to the patient.

Therefore, local, site specific delivery of PAR-1 antagonists will decrease site specific proliferation and/or migration of smooth muscle cells. The main benefits of local delivery of a PAR-1 antagonist would be comprised of increased intensity and duration of vasodiolatory response, increased vascular thromboresistance, decreased migration of smooth muscle cells, and inhibition of SMC proliferation.

In one embodiment, the PAR-1 antagonists are specific to thrombin. Suitable PAR-1 antagonists include but are not limited to peptide based antagonists such as Mpr-peptide, and synthetic antagonists such as RWJ-58259, BMS-200261 and SCH-530348. In one embodiment, a PAR-1 antagonist is provided such as, but not limited to, SCH-530348, as depicted below.

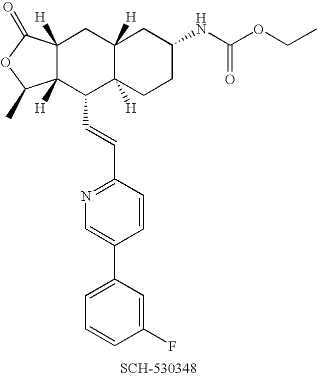

It will be understood by those skilled in the art, that the above formula is but one of many pharmaceutically acceptable PAR-1 antagonists. Many other pharmaceutically acceptable forms can be synthesized. Moreover, many derivatives are also possible that do not affect the efficacy or mechanism of action of the PAR-1 antagonists. Therefore, SCH-530348 and pharmaceutically acceptable derivatives, salts and combinations thereof are all encompassed by the description herein.

The PAR-1 antagonists discussed herein may be added to implantable medical devices. The PAR-1 antagonists may be incorporated into a polymer coating applied to the surface of a medical device or may be incorporated into a polymer used to form the medical device. The PAR-1 antagonist may be coated to the surface of the medical device with or without a polymer using methods including, but not limited to, precipitation, coacervation, and crystallization. The PAR-1 antagonist may be bound to the medical device or a coating on a medical device covalently, ionically, or through other intramolecular interactions, including without limitation, hydrogen bonding and van der Waals forces.

The medical devices used herein may be permanent medical implants, temporary implants, or removable devices. For example, and not intended as a limitation, the medical devices may include stents, catheters, micro-particles, probes, and vascular grafts.

In one embodiment, stents may be used as a drug delivery platform. The stents may be vascular stents, urethral stents, biliary stents, or stents intended for use in other ducts and organ lumens. Vascular stents, for example, may be used in peripheral, neurological, or coronary applications. The stents may be rigid expandable stents or pliable self-expanding stents. Any biocompatible material may be used to fabricate the stents, including, without limitation, metals and polymers. The stents may also be bioresorbable. In one embodiment, vascular stents are implanted into coronary arteries immediately following angioplasty.

In one embodiment, metallic vascular stents are coated with one or more PAR-1 antagonists, including but not limited to, SCH-530348. The PAR-1 antagonist may be dissolved or suspended in any carrier compound that provides a stable, un-reactive environment for the antagonist. The stent can be coated with a PAR-1 antagonist coating according to any technique known to those skilled in the art of medical device manufacturing. Suitable non-limiting examples include impregnation, spraying, brushing, dipping and rolling. After the PAR-1 antagonist is applied to the stent, it is dried leaving behind a stable PAR-1 antagonist delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the PAR-1 antagonists.

The effective amount of PAR-1 antagonist can be determined by a titration process. Titration is accomplished by preparing a series of stent sets. Each stent set will be coated, or contain different dosages of PAR-1 antagonist. The highest concentration used will be partially based on the known toxicology of the compound. The maximum amount of drug delivered by the stents will fall below known toxic levels. The dosage selected for further studies will be the minimum dose required to achieve the desired clinical outcome. The desired clinical outcome is defined as a site specific decrease in smooth muscle cell proliferation and/or a decrease in smooth muscle cell migration.

In another embodiment, the PAR-1 antagonist is precipitated or crystallized onto or within the stent. In yet another embodiment, the PAR-1 antagonist is mixed with a suitable biocompatible polymer (bioerodable, bioresorbable, or non-erodable). The polymer-PAR-1 antagonist blend can then be used to produce a medical device such as, but not limited to, stents, grafts, micro-particles, sutures and probes. Furthermore, the polymer-PAR-1 antagonist blend can be used to form controlled-release coatings for medical device surfaces. For example, and not intended as a limitation, the medical device can be immersed in the polymer-PAR-1 antagonist blend, the polymer-PAR-1 antagonist blend can be sprayed, or the polymer-PAR-1 antagonist blend can be brushed onto the medical device. In another embodiment, the polymer-PAR-1 antagonist blend can be used to fabricate fibers or strands that are embedded into the medical device or used to wrap the medical device.

In one embodiment, the polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; polyvinyl amides; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer coatings or medical devices formed from polymeric material discussed herein may be designed with a specific dose of PAR-1 antagonist. That dose may be a specific weight of antagonist added or a PAR-1 antagonist to polymer ratio. In one embodiment, the medical device can be loaded with from about 1 to about1000 μg of PAR-1 antagonist; in another embodiment, from about 5 μg to about 500 μg; in another embodiment from about 10 μg to about 250 μg; in another embodiment, from about 15 μg to about150 μg. A ratio may also be established to describe how much PAR-1 antagonist is added to the polymer that is coated to or formed into the medical device. In one embodiment a ratio of 1 part PAR-1 antagonist: 1 part polymer may be used; in another embodiment, from about 1:1 to about 1:5; in another embodiment, from about 1:1 to about 1:9; in another embodiment, from about 1:1 to about 1:20.

In addition to the site specific delivery of PAR-1 antagonists, the implantable medical devices discussed herein can accommodate one or more additional bioactive agents. The choice of bioactive agent to incorporate, or how much to incorporate, will have a great deal to do with the polymer selected to coat or form the implantable medical device. A person skilled in the art will appreciate that hydrophobic agents prefer hydrophobic polymers and hydrophilic agents prefer hydrophilic polymers. Therefore, coatings and medical devices can be designed for agent or agent combinations with immediate release, sustained release or a combination of the two.

Exemplary, non limiting examples of bioactive agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers.

### EXAMPLES

The following Examples are intended to illustrate a non-limiting process for coating metallic stents with a PAR-1 antagonist. One non-limiting example of a metallic stent suitable for use in accordance with the teachings described herein is the Medtronic/AVE S670™ 316L stainless steel coronary stent.

### Example 1

### Metal Stent Cleaning Procedure

Stainless steel stents were placed a glass beaker and covered with reagent grade or better hexane. The beaker containing the hexane immersed stents was then placed into an ultrasonic water bath and treated for 15 minutes at a frequency of between approximately 25 to 50 KHz. Next the stents were removed from the hexane and the hexane was discarded. The stents were then immersed in reagent grade or better 2-propanol and vessel containing the stents and the 2-propanol was treated in an ultrasonic water bath as before. Following cleaning the stents with organic solvents, they were thoroughly washed with distilled water and thereafter immersed in 1.0 N sodium hydroxide solution and treated at in an ultrasonic water bath as before. Finally, the stents were removed from the sodium hydroxide, thoroughly rinsed in distilled water and then dried in a vacuum oven over night at 40° C. After cooling the dried stents to room temperature in a desiccated environment they were weighed their weights were recorded.

### Example 2

### Coating a Clean, Dried Stent using a Bioactive Agent/Polymer System

In the following Example, ethanol is chosen as the solvent of choice. The PAR-1 antagonist is SCH-530348. Both the polymer and SCH-530348 are freely soluble ion ethanol. Persons having ordinary skill in the art of polymer chemistry can easily pair the appropriate solvent system to the polymer-drug combination and achieve optimum results with no more than routine experimentation.

250 mg of SCH-530348 is carefully weighed and added to a small neck glass bottle containing 2.8 ml of ethanol. The SCH-530348-ethanol suspension is then thoroughly mixed until a clear solution is achieved.

Next 250 mg of polycaprolactone (PCL) is added to the SCH-530348-ethanol solution and mixed until the PCL dissolved forming a drug/polymer solution.

The cleaned, dried stents are coated using either spraying techniques or dipped into the drug/polymer solution. The stents are coated as necessary to achieve a final coating weight of between approximately 10 μg to 1 mg. Finally, the coated stents are dried in a vacuum oven at 50° C. overnight. The dried, coated stents are weighed and the weights recorded.

The concentration of bioactive agent loaded onto (into) the stents is determined based on the final coating weight. Final coating weight is calculated by subtracting the stent's pre-coating weight from the weight of the dried, coated stent.

### Example 3

### Coating a Clean, Dried Stent using a Sandwich-Type Coating

A cleaned, dry stent is first coated with polyvinyl pyrrolidone (PVP) or another suitable polymer followed by a coating of SCH-530348. Finally, a second coating of PVP is provided to seal the stent thus creating a PVP- SCH-530348-PVP sandwich coated stent.

### The Sandwich Coating Procedure:

100 mg of PVP is added to a 50 mL Erlenmeyer containing 12.5 ml of ethanol. The flask was carefully mixed until all of the PVP is dissolved. In a separate clean, dry Erlenmeyer flask 250 mg of SCH-530348 is added to 11 mL of ethanol and mixed until dissolved.

A clean, dried stent is then sprayed with PVP until a smooth confluent polymer layer was achieved. The stent was then dried in a vacuum oven at 50° C. for 30 minutes.

Next, successive layers of SCH-530348 are applied to the polymer-coated stent. The stent is allowed to dry between each of the successive SCH-530348 coats. After the final SCH-530348 coating has dried, three successive coats of PVP are applied to the stent followed by drying the coated stent in a vacuum oven at 50° C. over night. The dried, coated stent is weighed and its weight recorded.

The concentration of bioactive agent in the bioactive agent/polymer solution and the final amount of bioactive agent loaded onto the stent determine the final coating weight. Final coating weight is calculated by subtracting the stent's pre-coating weight from the weight of the dried, coated stent.

### Example 4

### Coating a Clean, Dried Stent with Pure Drug

1.00 g of SCH-530348 is carefully weighed and added to a small neck glass bottle containing 12 ml of ethanol. The SCH-530348-ethanol suspension is then heated at 50° C. for 15 minutes and then mixed until the SCH-530348 is completely dissolved.

Next a clean, dried stent is mounted over the balloon portion of angioplasty balloon catheter assembly. The stent is then sprayed with, or in an alternative embodiment, dipped into, the SCH-530348-ethanol solution. The coated stent is dried in a vacuum oven at 50° C. overnight. The dried, coated stent was weighed and its weight recorded.

The concentration of bioactive agent loaded onto (into) the stents is determined based on the final coating weight. Final coating weight is calculated by subtracting the stent's pre-coating weight from the weight of the dried, coated stent.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A medical device system for treating vascular conditions comprising an implantable device for the site-specific, controlled delivery of a therapeutic amount of a PAR-1 antagonist.

2. The medical device system according to claim 1 wherein said PAR-1 antagonist has a molecular structure depicted below, 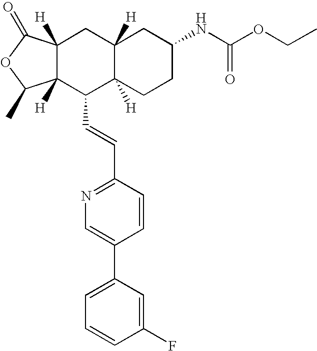
and pharmaceutically acceptable derivatives, salts, and combinations thereof.

3. The medical device system according to claim 2 wherein said PAR-1 antagonist is SCH-530348.

4. The medical device system according to any of claims 2 or 3 wherein said medical device is selected from the group consisting of stents, catheters, micro-particles, probes and vascular grafts.

5. The medical device system according to claim 4 wherein said stent is a vascular stent.

6. The medical device system according to claim 5 wherein said vascular stent is provided with a coating comprising SCH-530348, pharmaceutically acceptable derivatives, salts, or combinations thereof.

7. The medical device system according to claim 6 wherein said coating further contains a biocompatible polymer.

8. The medical device system according to claim 1 wherein said medical device comprises at least one additional drug, said at least one additional drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

9. The medical device system according to claim 1 further comprising at least one additional drug selected from the group consisting of sirolimus (rapamycin), tacrolimus (FK506), everolimus (certican), temsirolimus (CCI-779) and zotarolimus (ABT-578).

10. The medical device system according to claim 7 wherein said coating comprises:
between about 50 μg and about 250 μg of a PAR-1 antagonist, and a polymer, wherein said PAR-1 antagonist and said polymer are in a ratio relative to each other of 1 part PAR-1 antagonist to between about 1 and about 9 parts polymer; and
wherein said PAR-1 antagonist comprises SCH-530348, pharmaceutically acceptable derivatives, salts, or combinations thereof.

11. A method of treating or inhibiting vascular conditions comprising:
(a) providing an implantable medical device,
(b) providing a PAR-1 antagonist;
(c) providing at least one biocompatible polymer;
(d) dispersing said PAR-1 antagonist within said at least one polymer thereby forming a drug loaded polymer;
(d) coating at least a portion of said medical device with said drug loaded polymer; and
(e) implanting said medical device into a blood vessel lumen wherein said PAR-1 antagonist is released into tissue adjacent said blood vessel lumen.

12. The method according to claim 11 wherein said coating comprises:
between about 50 μg and about 250 μg of a PAR-1 antagonist, and a polymer, wherein said PAR-1 antagonist and said polymer are in a ratio relative to each other of 1 part PAR-1 antagonist to between about 1 and about 9 parts polymer;
wherein said PAR-1 antagonist comprises SCH-530348, pharmaceutically acceptable derivatives, salts, or combinations thereof.

13. The method according to claim 11 wherein said medical device is a vascular stent.

14. The method according to claim 11 wherein said medical device comprises at least one additional drug selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

15. The method according to claim 11 wherein said at least one additional drug is selected from the group consisting of sirolimus (rapamycin), tacrolimus (FK506), everolimus (certican), temsirolimus (CCI-779) and zotarolimus (ABT-578).

16. A method of treating or inhibiting restenosis comprising:
providing a vascular stent having a coating comprising SCH-530348, pharmaceutically acceptable derivatives, salts, or combinations thereof, and at least one additional drug selected from the group consisting of antiplatelet agents, antimigratory agent, antifibrotic agents, antiproliferatives, antiinflammatories and combinations thereof; and
implanting said vascular stent into a blood vessel lumen wherein said SCH-530348, pharmaceutically acceptable derivatives, salts, or combinations thereof, and at least one additional drug, is released into tissue adjacent to said blood vessel lumen.
